(19) United States
(12) Patent Application Publication
(10) Pub. No.: US 2022/0054177 A1
(43) Pub. Date: Feb. 24, 2022

(54) IMPLANT HOLDER

(71) Applicant: Medos International Sarl, Le Locle (CH)

(72) Inventors: Kristina Carlson, Hope Valley, RI (US); Catherine Britan, Raynham, MA (US)

(21) Appl. No.: 17/000,965

(22) Filed: Aug. 24, 2020

(51) Int. Cl. A61B 17/86; A61B 17/70; A61B 50/30

(52) U.S. Cl. CPC A61B 17/865; A61B 50/30; A61B 17/7035

(57) ABSTRACT

Various implant holding systems are provided herein that include an outer tube, an inner tube, and a holder. The implant holding systems can be designed to hold various implants, such as one or more set screws used in spinal operations. For example, one embodiment of an implant holding system can have an outer tube, an inner tube that can be disposed within the outer tube, and a surgical implant holder that can be disposed within the inner tube. One or more stackable surgical implants, such as set screws, of various configurations and sizes can be disposed within the holder, and the holder can engage the surgical implants to assist in maintaining an orientation of the surgical implants relative to the holder.

### FIELD

The present disclosure relates generally to implant holders, such as set screw holders.

### BACKGROUND

During various surgical operations, it is necessary to maintain a surgically sterile environment. As such, preparing for operations that require multiple tools and implants, such as spinal operations in which a plurality of bone anchors and set screws may be used, can be very time consuming because a user is required to sterilize a number of different implants. Additionally, many surgical implants, such as set screws, are provided to users on a large tray with numerous different sizes and configurations. A user is required to sterilize the entire tray even if an operation only requires a small number of the surgical implants on the tray, and a user must pay for, maintain, and ensure proper training is provided for any implant sterilization equipment. Furthermore, having to manage large containers of surgical implants further complicates surgeries, leads to disorder and clutter in the operating room, and requires significant storage space.

Accordingly, there remains a need for improved implant holders.

### SUMMARY

In general, implant holders and methods for using the same are provided.

In one embodiment, a surgical holding system is provided that has an outer tube, an inner tube, a holder, and one or more surgical implants. The outer tube has a sealed first outer end and an open second outer end, and the outer tube has a removable outer cap that is configured to selectively seal the second outer end. The inner tube is configured to be disposed within the outer tube, and the inner tube has a sealed first inner end that is configured to abut against the outer cap of the outer tube. The inner tube also has an open second inner end and a removable inner cap that is configured to selectively seal the second inner end. The holder is configured to be disposed within the inner tube and has a longitudinal axis. Furthermore, the one or more surgical implants can be a plurality of surgical implants, and the holder is configured to engage the plurality of surgical implants such that the surgical implants are held stationary within and relative to the inner tube.

The system can have numerous variations. For example, the plurality of surgical implants can be positioned along the longitudinal axis of the holder. In another example, the holder can have at least one cavity formed therein that is configured to receive the plurality of surgical implants. In another example, the holder can have a window that extends through a sidewall of the holder from an outer surface of the holder to the at least one cavity such that the plurality of surgical implants is visible through the window. The holder can also have a plurality of markings on the outer surface thereof that extend along an edge of the window, and the plurality of markings can be configured to provide information about the plurality of surgical implants held in the holder. The at least one cavity can also comprise a cylindrical cavity and can extend along the longitudinal axis of the holder. In still another example, the holder can have an engagement post that extends along the longitudinal axis and into the cylindrical cavity, and the engagement post can be configured to extend through an opening in each of the plurality of surgical implants when the plurality of surgical implants are in engagement with the holder. In some examples, the engagement post can have alignment features thereon, and the alignment features can be configured to orient the plurality of surgical implants in a shared direction. In still another example, the engagement post can be configured to be at least partially retractable out of the cylindrical cavity and into the holder during loading and unloading of the plurality of surgical implants. In one example, the holder can have a plurality of spacing lips that extend into the cylindrical cavity, and each of the plurality of spacing lips can be configured to prevent the plurality of surgical implants from contacting each other. In some examples, the at least one cavity can comprise a plurality of cavities, and each of the plurality of cavities can be configured to receive one of the plurality of surgical implants therein. In other examples, a cross section of the holder along the longitudinal axis can be one of a circle or a rectangle. The outer tube, the inner tube, and the holder can also be configured to be coaxial with each other along the longitudinal axis of the holder when the holder is disposed within the inner tube and the inner tube is disposed within the outer tube. The plurality of surgical implants can also include one of a plurality of set screws or a plurality of nuts.

In another embodiment, an implant holding system is provided that has an outer tube, an inner tube, a holder, and one or more surgical implants. The outer tube has a sealed first outer end and an open second outer end, and the outer tube has a removable outer cap that is configured to selectively seal the second outer end. The inner tube is configured to be disposed within the outer tube, and the inner tube has a sealed first inner end that is configured to contact the outer cap of the outer tube. The inner tube also has an open second inner end and a removable inner cap that is configured to selectively seal the second inner end. The holder is configured to be disposed within the inner tube. The holder also has a longitudinal axis that extends between first and second holder ends, and a lumen extends at least partially through the holder from the second holder end toward the first holder end. Furthermore, the one or more surgical implants can be a plurality of surgical implants, and the lumen of the holder is configured to receive the plurality of surgical implants therein in an engagement fit such that an orientation of each of the plurality of surgical implants relative to the holder is maintained when the plurality of surgical implants are received in the lumen.

The system can have numerous variations. For example, the plurality of surgical implants can comprise one of a plurality of set screws or a plurality of nuts. In another example, an engagement post can extend along the longitudinal axis of the holder at least partially into the lumen from the first holder end toward the second holder end, and the engagement post can be configured to engage each of the plurality of surgical implants. In still another example, the plurality of surgical implants can be positioned along the longitudinal axis of the holder. In some examples, the holder can have a window that extends through a sidewall of the holder from an outer surface of the holder to the lumen such that the plurality of surgical implants is visible through the window.

In another aspect, a surgical method is provide that includes removing a sealed cover from an outer tube while the outer tube houses an inner tube. A surgical holder is disposed within the inner tube, and a plurality of surgical implants are held in a first orientation within a lumen of the surgical holder relative to a longitudinal axis of the surgical holder. The plurality of surgical implants are held through direct engagement between the surgical holder and each of the plurality of surgical implants, and an interior of the outer tube, the inner tube, the implant holder, and the plurality of surgical implants are sterile. The method also includes passing the inner tube into a sterile field without contaminating an exterior surface of the inner tube and removing a cover from the inner tube within the sterile field. The method further includes retrieving at least one of the plurality of surgical implants from the surgical holder in the inner tube within the sterile field while the plurality of surgical implants remain in the first orientation during retrieval through engagement with the surgical holder.

The method can have numerous variations. For example, the method can further include visually inspecting an orientation of the plurality of surgical implants through the outer tube, the inner tube, and the surgical holder during removal of the sealed cover from the outer tube. In another example, retrieving at least one of the plurality of surgical implants can include inserting a loading device at least partially into the inner tube and loading the at least one set screw onto the loading device. The plurality of surgical implants can also include one of a plurality of set screws or a plurality of nuts.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

This invention will be more fully understood from the following detailed description taken in conjunction with the accompanying drawings, in which:

FIG. 1A is a perspective view of one embodiment of a surgical implant holder and one embodiment of a plurality of surgical implants disposed within one embodiment of an inner tube, which is disposed within one embodiment of an outer tube;

FIG. 1B is a perspective view of the implant holder, the plurality of surgical implants, and the inner tube of FIG. 1A;

FIG. 1C is a perspective view of the implant holder of FIG. 1A;

FIG. 1D is a perspective view of the implant holder, the plurality of surgical implants, and the inner tube of FIG. 1A;

FIG. 1E is a perspective view of the implant holder, the plurality of surgical implants, and the inner tube of FIG. 1A;

FIG. 1F is a perspective view of the implant holder, the plurality of surgical implants, and the inner tube of FIG. 1A;

FIG. 2 is a perspective view of another embodiment of a surgical implant holder;

FIG. 3 is a perspective view of another embodiment of a surgical implant holder;

FIG. 4 is a perspective view of another embodiment of a surgical implant holder with another embodiment of a plurality of surgical implants;

FIG. 5 is a perspective view of the surgical implant holder and the plurality of surgical implants of FIG. 4;

FIG. 6 is a diagram of one embodiment of steps for passing the inner tube, the surgical implant holder, and the plurality of surgical implants of FIG. 1A into a sterile environment;

FIG. 7 is another diagram of the steps of FIG. 6 for passing the inner tube, the surgical implant holder, and the plurality of surgical implants of FIG. 1A into a sterile environment;

FIG. 8A is a perspective view of another embodiment of a surgical implant holder retaining the plurality of surgical implants of FIG. 1A;

FIG. 8B is a cross-sectional perspective view of the surgical implant holder of FIG. 8A with the plurality of surgical implants of FIG. 1A;

FIG. 8C is a top down view of the surgical implant holder of FIG. 8A with the plurality of surgical implants of FIG. 1A;

FIG. 9A is a partially transparent perspective view of another embodiment of a surgical implant holder retaining the plurality of surgical implants of FIG. 1A;

FIG. 9B is a cross-sectional perspective view of the surgical implant holder of FIG. 9A with the plurality of surgical implants of FIG. 1A;

FIG. 9C is a cross-sectional side view of the surgical implant holder of FIG. 8A with the plurality of surgical implants of FIG. 1A;

FIG. 9D is a partially transparent perspective view of the surgical implant holder of FIG. 9A with a shaft of one embodiment of a surgical insertion device engaging the plurality of surgical implants of FIG. 1A;

FIG. 9E is a cross-sectional perspective view of the surgical implant holder of FIG. 9A with the shaft of the surgical insertion device of FIG. 9D engaging the plurality of surgical implants of FIG. 1A;

FIG. 9F is a cross-sectional side view of the surgical implant holder of FIG. 9A with the shaft of the surgical insertion device of FIG. 9D engaging the plurality of surgical implants of FIG. 1A;

FIG. 9G is a partially transparent perspective view of the surgical implant holder of FIG. 9A with the shaft of the surgical insertion device of FIG. 9D removing the plurality of surgical implants of FIG. 1A;

FIG. 9H is a perspective view of the surgical implant holder of FIG. 9A disposed within the inner tube of FIG. 1A and the surgical insertion device of FIG. 9D engaging with one of the plurality of surgical implants of FIG. 1A;

FIG. 9I is a perspective view of the surgical implant holder of FIG. 9A disposed within the inner tube of FIG. 1A and the surgical insertion device of FIG. 9D removing one of the plurality of surgical implants of FIG. 1A;

FIG. 10A is a cross-sectional side view of an upper portion of another embodiment of a surgical implant holder retaining the plurality of surgical implants of FIG. 1A;

FIG. 10B is a cross-sectional perspective view of the upper portion of the surgical implant holder of FIG. 10A retaining the plurality of surgical implants of FIG. 1A;

FIG. 11 is a side view of another embodiment of a surgical implant holder retaining the plurality of surgical implants of FIG. 1A;

FIG. 12 is a side view of another embodiment of a surgical implant holder retaining the plurality of surgical implants of FIG. 1A;

FIG. 13A is a partially transparent perspective view of another embodiment of a surgical implant holder retaining one embodiment of a cartridge with the plurality of surgical implants of FIG. 1A;

FIG. 13B is a cross-sectional perspective view of the surgical implant holder and the cartridge of FIG. 13A with the plurality of surgical implants of FIG. 1A;

FIG. 14A is a perspective view of another embodiment of a surgical implant holder retaining the plurality of surgical implants of FIG. 1A;

FIG. 14B is another perspective view of the surgical implant holder of FIG. 14A with the plurality of surgical implants of FIG. 1A;

FIG. 15A is a partially transparent perspective view of another embodiment of a surgical implant holder;

FIG. 15B is a partially transparent perspective view of the surgical implant holder of FIG. 15A retaining the plurality of surgical implants of FIG. 1A;

FIG. 15C is a cross-sectional perspective view of the surgical implant holder of FIG. 15A;

FIG. 15D is a cross-sectional perspective view of the surgical implant holder of FIG. 15A retaining the plurality of surgical implants of FIG. 1A; and

FIG. 16 is a perspective view of another embodiment of an inner tube illustrated to scale next to the inner tube and the holder of FIG. 1A with one embodiment of a user's hand and a ruler for scale.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Sizes and shapes of the systems and devices, and the components thereof, can depend at least on the anatomy of the subject in which the systems and devices will be used, the size and shape of components with which the systems and devices will be used, and the methods and procedures in which the systems and devices will be used. Like reference symbols in the various drawings indicate like elements.

The present disclosure generally relates to surgical implant holders. When one or more implants are transported, such as a plurality of set screws, it can be helpful to maintain a general orientation of the transported implants to prevent the implants from being damaged during transportation and to minimize any difficulty in removing one or more of the implants from the transportation container during deployment, especially if the surgical implant needs to be loaded onto a loading or insertion device for deployment. Additionally, it can be beneficial to sterilize and package a predetermined number of implants to be used together in one transportation container so that, upon deployment, a user is not required to sterilize any of the implants and is not required to handle and potentially contaminate more implants than will be needed during a current operation.

As such, various surgical implant holding systems are provided herein that include an outer tube, an inner tube, and a surgical implant holder. The implant holding systems can be designed to accommodate various numbers of surgical implants that are different types and sizes, such as one or more set screws, nuts, washers, and other stackable components used in surgical operations. The surgical implant holder can engage the one or more surgical implants disposed therein to assist in maintaining an orientation of the surgical implants relative to the holder, and the holder can engage the inner tube to assist in maintaining an orientation of the holder within the inner tube. The inner tube, in turn, is maintained within an outer tube.

Additionally, one or more of the surgical implant(s), the holder, the inner tube, and/or the outer tube can be sterilized during assembly packaging, and maintained in a sterile state until the surgical implants are deployed. Typically, a predetermined number of surgical implants needed in a single operation, the holder, and the inner tube are sterilized before placement within a sterile lumen of the outer tube. This ensures that each surgical implant is sterilized and maintained within a sterile enclosure, making it possible only to open (and potentially contaminate) the surgical implants that will be needed in a current operation or surgical procedure without compromising the sterility of any other surgical implants not used during the procedure.

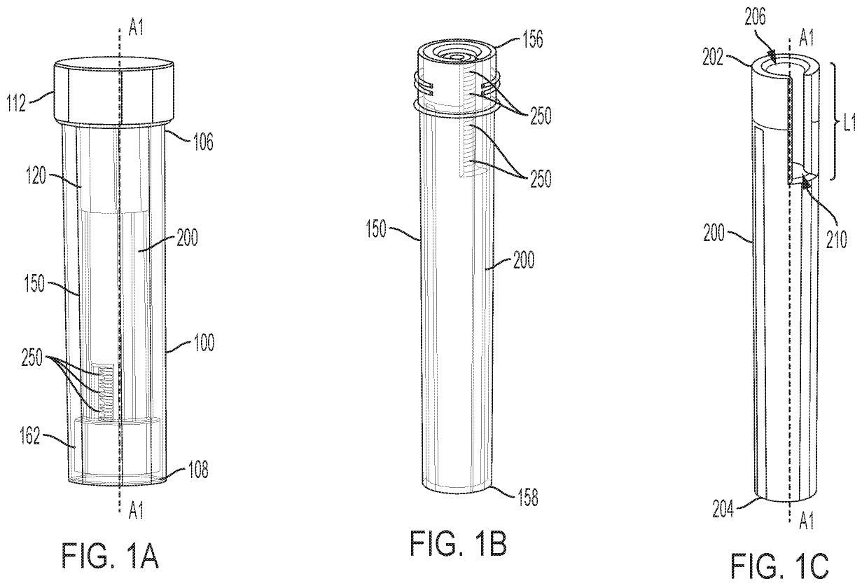

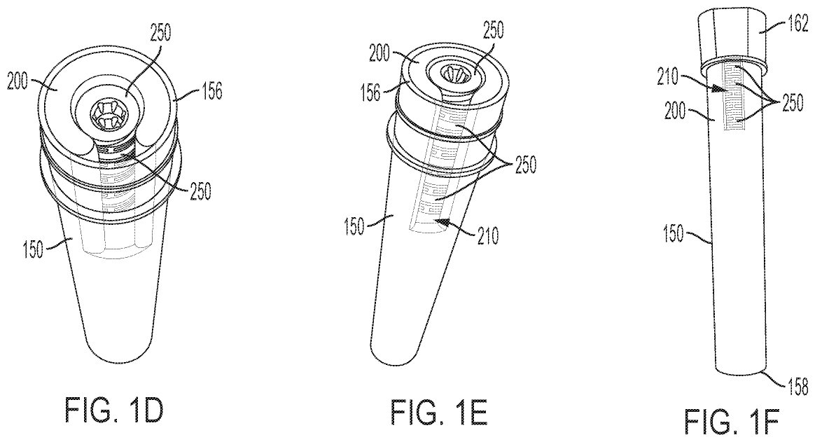

FIGS. 1A-1F illustrate one embodiment of a surgical implant holding system that has an outer tube 100, an inner tube 150, and a holder 200, as collectively illustrated in FIG. 1A. The holder 200 receives a plurality of surgical implants 250 therein so that orientations of the surgical implants 250 are maintained relative to the holder 200 during movement and transport of the system. As discussed further below, the holder 200 can be placed in a lumen of the inner tube 150 and can maintain a position within the inner tube 150. The inner tube 150 can be placed in a lumen of the outer tube 100, and the inner tube 150 can also maintain its position within the outer tube 100. As such, orientations of the surgical implants 250 can be maintained within the holder 200 and tubes 100, 150 during transportation, for example when being moved from a manufacturing or distribution facility to an operating environment in which the surgical implants 250 will be deployed.

While the holder 200 can have a variety of different shapes and sizes to maintain its position within the inner tube 150, as discussed further below, the holder illustrated in FIGS. 1A-1F is a generally cylindrical structure that is sized to substantially fill the lumen of the inner tube 150 when inserted therein, thus preventing the holder 200 from rotating or sliding inside the inner tube 150 during transportation because of the flush or near-flush fit. The holder 200 has a first end 202, a second end 204, a longitudinal axis A1 extending therebetween, and a lumen 206 and window 210 extending from the first end 202 partially toward the second end 204.

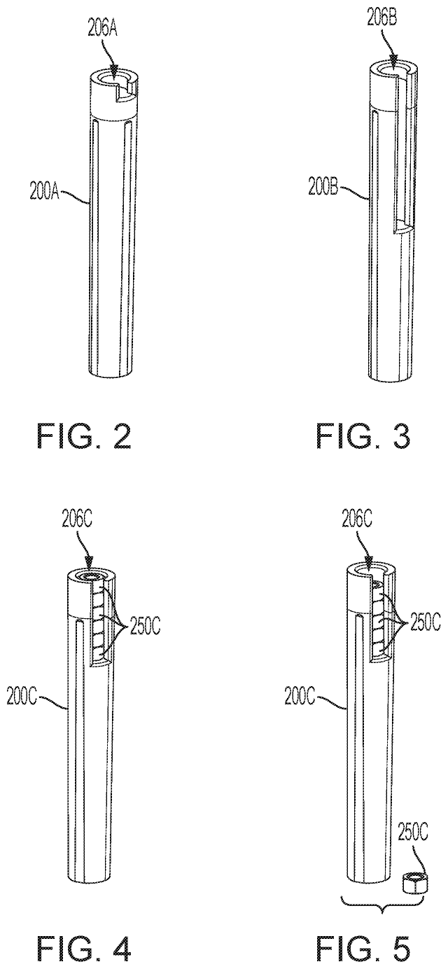

The lumen or cavity 206 extends along the longitudinal axis A1, and a cross-sectional shape and a length L1 of the lumen 206 along the axis A1 are selected to receive a plurality of the surgical implants 250 in a sequentially stacked orientation, as illustrated in FIG. 1B. The plurality of surgical implants 250 received in the lumen 206 is the number of implants intended to be used during a single operation so that a user does not have to re-sterilize any unused implants 250 after opening the inner tube 150 inside a sterile operating field, discussed below. As such, the length L1 of the lumen 206 along the axis Al can be varied in other embodiments to allow more or fewer implants 250 to be received therein depending on how many implants 250 are expected to be needed. The illustrated length L1 of the lumen 206 in FIGS. 1A-1F is selected to allow 8 surgical implants 250 to be received therein in the stacked orientation. However, FIG. 2 illustrates another embodiment of a surgical holder 200A, similar to holder 200, with a lumen 206A that can fit only a single surgical implant 250 therein, and FIG. 3 illustrates still another embodiment of a surgical holder 200B, similar to holder 200, with a lumen 206B that can fit sixteen surgical implants 250 therein. The length of the lumen 206 can thus vary in other embodiments to allow different numbers of stacked surgical implants 250, such as between 1 and 50 implants, and more specifically, 1 implant, 4 implants, 10 implants, 12 implants, 14 implants, 16 implants, 18 implants, 20 implants, etc.

Furthermore, the window or opening 210 is formed in a sidewall of the holder 200 and extends from the first end 202 along the entire length L1 of the lumen 206 to expose the lumen 206 and the surgical implants 250 therein. As such, a user can see the surgical implants 250 within the lumen 206 through the window 210 and can touch the implants 250 if needed, such as during loading or unloading. In other embodiments, the size and shape of the window can be varied, such as extending along only a portion of the lumen 206, not extending from the first end 202 such that the window is enclosed on all sides by sidewalls of the holder, etc. In other embodiments, a clear cover or screen can be placed over the window 210 to allow visualization of the implants 250 while blocking the window. In still other embodiments, the holder can exclude the window entirely and have an entirely solid sidewall.

As noted above, the illustrated cross sectional shape of the lumen 206 is circular to allow the illustrated surgical implants, set screws, to be received in the lumen 206 in a stacked orientation. The illustrated surgical implants 250 are set screws that can be used with a number of different surgical screw systems, such as the various surgical screw systems detailed in US Patent Pub. No. 2019/0150989 of Biester et al., entitled “Bone Anchor Assemblies and Related Instrumentation,” filed on Nov. 7, 2018, and the various surgical screw systems detailed in U.S. patent application Ser. No. 16/697,726 of Peterson, et al., entitled “Implant Holder,” filed on Nov. 27, 2019, which are both incorporated herein by reference in their entireties. However, the cross sectional shape and size of the lumen 206 can be varied to allow use with different set screws and with different types of surgical implants entirely, such as nuts, washers, and other stackable components. For example, FIGS. 4 and 5 illustrate another embodiment of a surgical holder 200C, similar to holder 200, with a lumen 206C that is sized and shaped to receive nuts 250C therein. Thus, various cross sectional shapes can include triangles, rectangles, hexagons, octagons, etc.

As illustrated in FIGS. 1A, 6, and 7, the holder 200 can be disposed within the inner tube 150, and the inner tube 150 in turn can be disposed within the outer tube 100. The inner tube 150 assists in maintaining sterility and an orientation of the holder 200 within the inner tube 150. As such, the inner tube 150 has an open end 156, a sealed or closed end 158, a lumen extending therebetween into which the holder 200 is inserted, and a cap 162 that closes the open end 156. The cap 162 is removably engageable with threading on the open end 156, and the cap 162 has a flat surface facing the lumen of the inner tube 150 (and the holder 200 when the holder 200 is inserted therein). As such, the flat inward-facing surface of the cap 162 assists in preventing the holder 200 from sliding or translating along the axis Al when the cap 162 is attached to the inner tube 150, for example abutting against or engaging the first end 202 of the holder 200 to keep the holder 200 secured. In other embodiments, additional engagement mechanisms and structures can be added to the cap 162 to securely engage the holder 200. Additionally, while the illustrated embodiment of the inner tube 150 utilizes threading to engage the cap 162, a variety of engagement mechanisms can be used, such as friction fit, adhesives, etc.

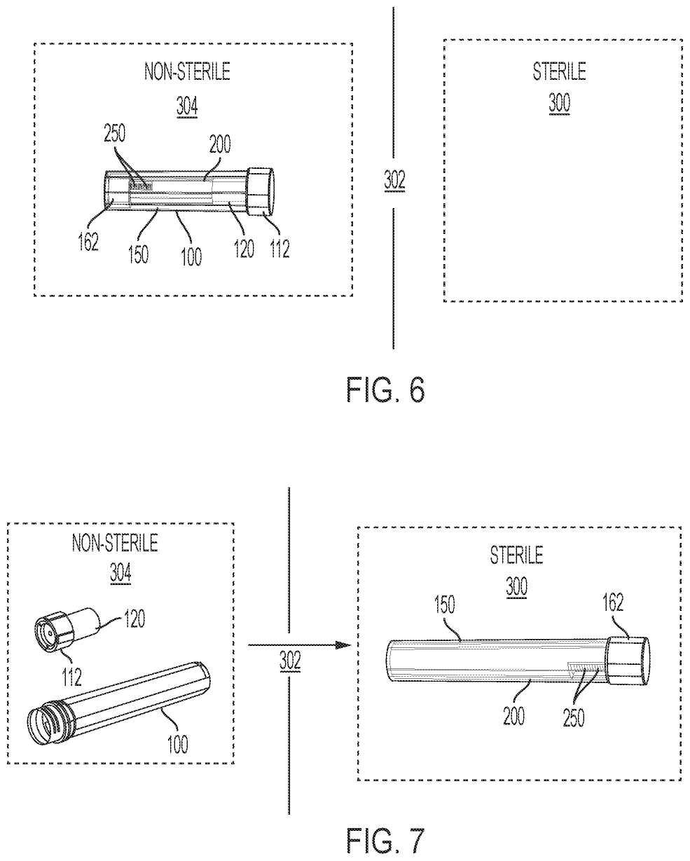

Additionally, as illustrated in FIGS. 1A and 6, the outer tube 100 can receive the inner tube 150 therein to assist in maintaining sterility and an orientation of the inner tube 150. Similar to the inner tube 150, the outer tube 100 has an open end 106, a sealed or closed end 108, a lumen extending therebetween into which the inner tube 150 can be inserted, and a cap 112 that closes the open end 106. The cap 112 is removably engageable with threading on the open end 106, and the cap 112 has an engagement interface 120 that protrudes from a surface of the cap 112 into the lumen of the outer tube 100 when the cap 112 closes the open end 106. The engagement interface 120 removably engages the closed end 158 of the inner tube 150 prior to the cap 112 being attached to the open end 106. As such, the inner tube 150 is held by and protrudes from the cap 112 of the outer tube 100 upon removal of the cap 112, and the inner tube 150 is thus maintained between the closed end 108 and the engagement interface 120 of the outer tube 100 when disposed within the outer tube 100. In some embodiments, the engagement interface 120 can be excluded such that the inner tube 150 can abut directly against the cap 112, and in still other embodiments, one or more additional spacers can be inserted into the lumen of the outer tube 100, for example against an inner surface of the closed end 108, to provide increased engagement of the inner tube 150 within the outer tube 100. Furthermore, while the illustrated embodiment of the outer tube 100 utilizes threading to engage the cap 112, a variety of engagement mechanisms can be used, such as friction fit, adhesives, etc.

Either one or both of the inner and outer tubes 100, 150 can also allow a user to visualize and observe an orientation of the surgical implants 250 through the tubes 100, 150 and the window 210 of the holder 200. For example, at least a portion of the inner tube 150 and at least a portion of the outer tube 100 are made from an optically transparent material. A user can thus ensure proper orientation of the surgical implants 250 has been maintained during movement and can confirm the contents of the tubes 100, 150 without being required to open the caps and sacrifice any sterile environment therein.

Furthermore, the tubes 100, 150, the holder 200, and the various components can be manufactured in a variety of ways and of a variety of materials. For example, the tubes 100, 150 and/or the holder 200 can be manufactured through injection molding, 3D printing, machining, etc., and can be made from various plastics, polymers, foams, rubbers, metals, etc. The materials used for each component can also be transparent or opaque.

In use, as illustrated in FIGS. 6 and 7, a user can insert the plurality of surgical implants 250 into the holder 200. The holder 200 can be inserted into the lumen of the inner tube 150, and the holder 200 substantially fills the lumen of the inner tube 150 to maintain the orientation of the holder 200, and consequently, the surgical implants 250 relative to the inner tube 150. The cap 162 is placed on the open end 156 of the inner tube 150 to enclose the holder 200 and the surgical implants 250. The closed end 158 of the inner tube 150 is placed in engagement with the engagement mechanism 120 of the cap 112, and the inner tube 150 and the engagement mechanism 120 are inserted into the lumen of the outer tube 100 through the open end 106. The cap 112 is placed on the open end 106 of the outer tube 100 to enclose the inner tube 150. The outer tube 100, the inner tube 150, and the holder 200 can all thus be coaxial once the implant holding system is assembled. Various labels, seals, tamper-proof closures, etc. can be applied to the outer tube 100, as desired, and the implant holding system is thus prepared for transportation and eventual deployment of the surgical implants 250.

Additionally, at any point during the manufacture and assembly process, one or more components of the system can be sterilized. For example, the outer tube 100, inner tube 150, holder 200, and surgical implants 250 can be sterilized and assembled in a sterile environment. As such, the holder 200 and the surgical implants 250 can be placed in a sterile environment within the lumen of the inner tube 150, and the inner tube 150 can then be placed within a sterile environment within the lumen of the outer tube 100. When the outer tube 100 is sealed and transported to a deployment site, such as an operating room, outer surfaces of the outer tube may have been contaminated and are no longer sterile. However, an interior environment of the outer tube 100, the inner tube 150, the holder 200, and the surgical implants 250 are all still sterile. Thus during deployment, a sterile operating environment 300 can be prepared, and an opening or passageway 302 between an external, non-sterile environment 304 and the sterile environment 300 can be formed, as illustrated in FIGS. 6 and 7. A user in the non-sterile environment 304 can select an implant system with the desired number of surgical implants 250, open the outer tube 100 of the selected system, and pass or drop the inner tube 150 through the passageway 302 into the sterile environment 300.

A user in the sterile environment 300 can then open the inner tube 150 within the sterile environment 300 and extract the surgical implants 250 without having to sterilize the implants 250 because the sterility of the inner tube 150, the holder 200, and the implants 250 have all been maintained. Additionally, because the number of surgical implants 250 can be selected beforehand, the user can deploy an exact number and type of surgical implants actually needed during a current operation without having to sterilize a large number of additional surgical implants and/or without having to store, maintain, and utilize any excess sterilization equipment and/or surplus surgical implants. Furthermore, because the holder 200 has maintained an orientation of the surgical implants 250 during transportation, the surgical implants 250 can be easily removed from the inner tube 150 and the holder 200 when the cap 162 is removed, for example manually or through use of various loading and insertion tools, such as the tools discussed in US Patent App. No. 2019/0150989 of Biester et al., identified and incorporated by reference above. Moreover, the inner tube 150 and holder 200 allow set screws to be packaged separately from other components used in various spinal operations, such as polyaxial screws. Because set screws are used at a later stage in many spinal surgeries than polyaxial screws, the set screws can remain sealed in the inner tube 150 until time of use, helping to avoid contamination and reduce clutter in the operating room.

Use of the holder 200 and the inner and outer tubes 100, 150 thus allows for a smaller storage footprint in the limited storage space outside of the operating room, less waste from excess packaging, increased organization in the operating room, and increased speed and ease of delivering implants to a surgeon. For example, currently, set screws are often individually packaged. During an operation requiring 8 set screws, a user needs to store, open, and handle 8 separate packages. Using the illustrated holder 200, the same user may only need to store, open, and handle only one package. Additionally, while the illustrated outer tube, inner tube, and holder are disposable, one or more of the outer tube, the inner tube, and the holder can be returned for re-sterilization and reuse in other embodiments, further reducing waste.

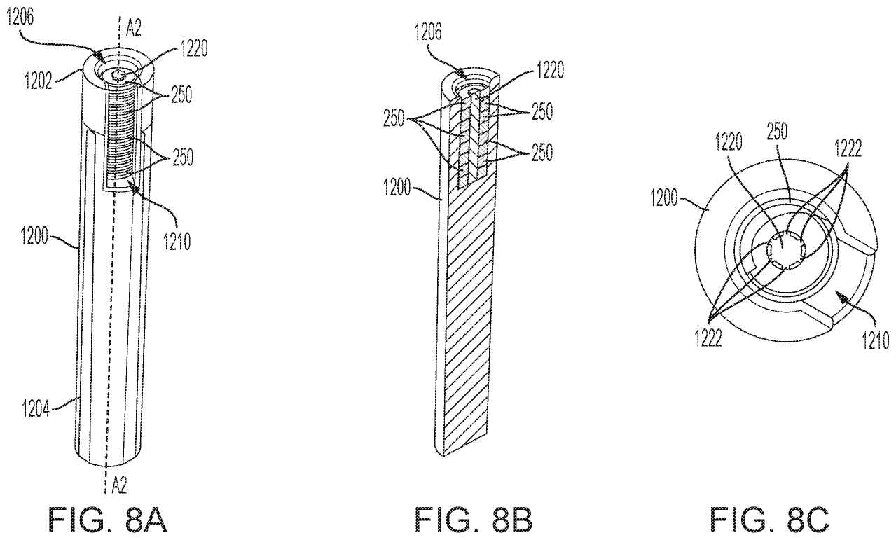

A number of other embodiments of surgical holders can be used in addition to or instead of the holder 200. For example, holders can assist in clocking or aligning implants and in loading implants onto various loading and insertion devices. FIGS. 8A-8C illustrate another embodiment of a holder 1200, similar to holder 200, with first and second ends 1202, 1204, a lumen 1206 extending partially therebetween, and a window 1210. The plurality of surgical implants 250 can be received in the lumen 1206, and the holder 1200 can be placed within inner and outer tubes 100, 150, as discussed above. An engagement post 1220 additionally extends from the holder 1200 and into the lumen 1206 toward the first end 1202 along a longitudinal axis A2 to assist with implant alignment. As the plurality of surgical implants 250 are inserted into the lumen 206, the engagement post 1220 passes through central openings in the surgical implants 250, as illustrated in FIG. 8B, so that the surgical implants 250 are engaged with and aligned on the engagement post 1220. Additionally, the engagement post 1220 has alignment surface features 1222 on outer surfaces thereof that can engage corresponding surface features in the central openings of the surgical implants 250. As the implants 250 are inserted onto the engagement post 1220, the surface features 1222 will act to clock or align all of the surgical implants 250 with each other to have the same orientation. During later removal of the implants, the surgical implants 250 will consequently all be oriented in the same direction, simplifying removal of the implants and any loading onto an insertion device. In other embodiments, however, surface features can be excluded, and a variety of different cross sectional shapes can be used for the engagement posts to ensure any desired engagement and alignment with various different surgical implants.

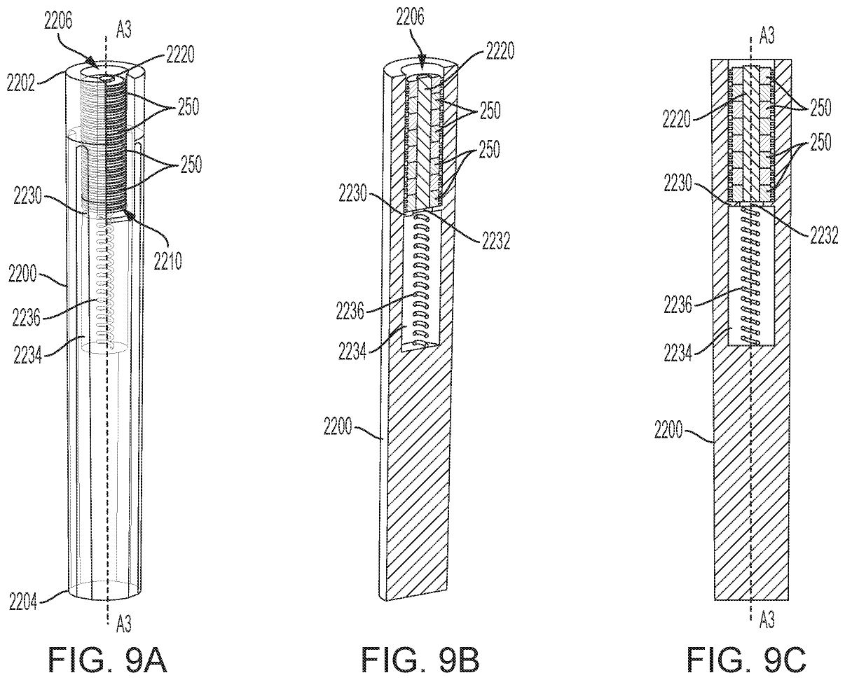

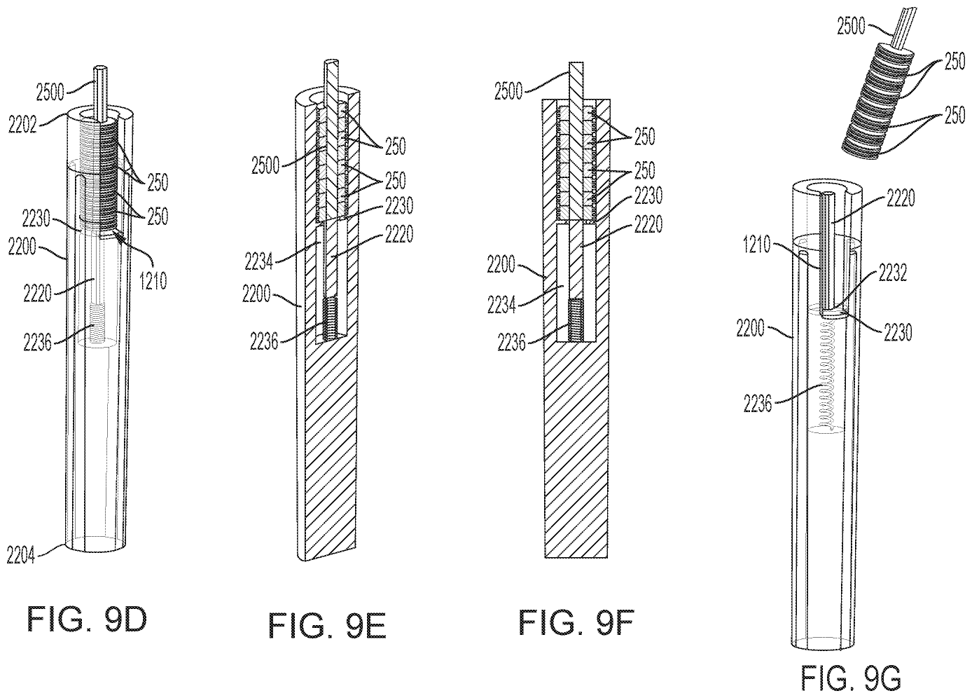

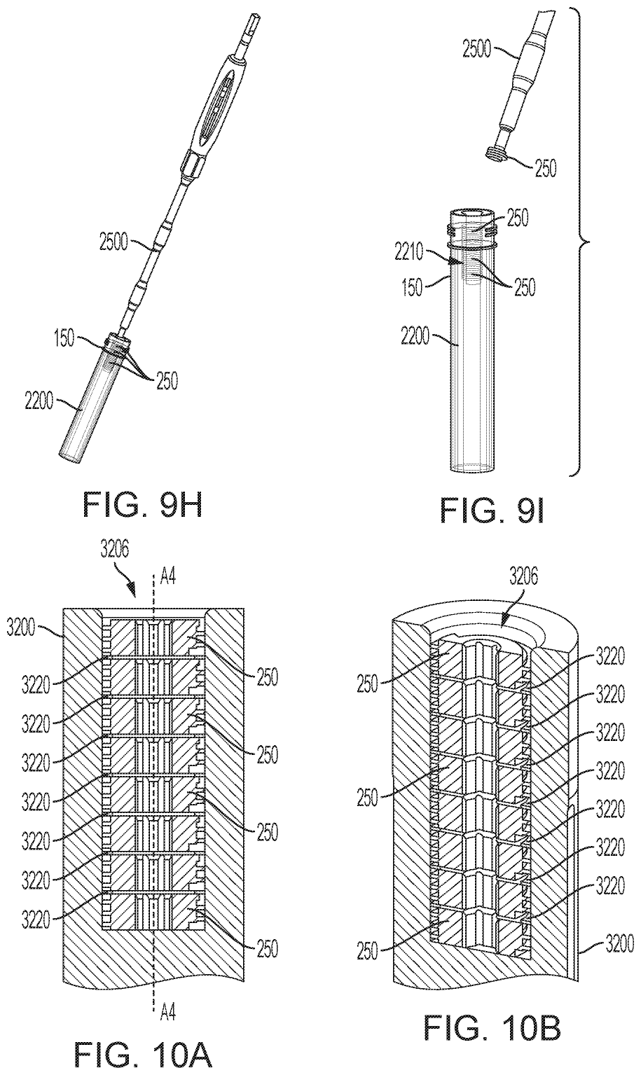

While the illustrated engagement post 1220 is fixed in position within the lumen 1206, the engagement post can be retractable in other embodiments to assist in loading implants onto an insertion device. For example, FIGS. 9A-9I illustrate another embodiment of a holder 2200, similar to holder 1200, with first and second ends 2202, 2204, a lumen 2206 extending partially therebetween, and a window 2210. The plurality of surgical implants 250 can be received in the lumen 2206 and around an engagement post 2220, similar to engagement post 1220.

The engagement post 2220 is additionally retractable out of the lumen 2206 when a force is applied to the post 2220, such as when a loading or insertion device is inserted into the lumen 2206 and passed through the central openings of the surgical implants 250 during loading. A ledge 2230 is formed at an end of the lumen 2206 opposite the first end 2202 of the holder 2200, and a cavity 2234 extends at least partially along a longitudinal axis A3 between the ledge 2230 and the second end 2204 of the holder 2200. A compression mechanism 2236, such as a spring, extends along the axis A3 through the cavity 2234. One end of the spring 2236 is embedded in the holder 2200 close to the second end 2204, and an opposite end of the spring 2236 engages with an end of the engagement post 2220 through an opening 2232 in the ledge 2230. As force is exerted on the engagement post 2220 along the axis A3 toward the second end 2204 of the holder 2200, the force compresses the spring 2236 and causes the engagement post 2220 to retract through the opening 2232 of the ledge 2230 and into the cavity 2234.

As illustrated in FIGS. 9D-91, a shaft of a loading or insertion tool 2500 can be aligned with and forced against the engagement post 2220. As the engagement post 2220 retracts into the cavity 2234 of the holder 2200, the shaft of the loading device 2500 passes through the central openings of one or more of the surgical implants 250, and the implants are loaded thereon. The loading device 2500 can then be removed from the lumen 2206 with one or more of the implants 250, as illustrated in FIGS. 9G and 91, and the engagement post 2220 can return to its pre-retraction position in the lumen 2206.

In still other embodiments, holders can reduce contact between the plurality of surgical implants when in the stacked orientation to limit scratching and other contact damage between sequential implants. FIGS. 10A and 10B illustrate a surgical holder 3200, similar to holder 200, that has first and second ends, a lumen 3206 extending partially therebetween, and a window. A plurality of alignment lips or ribs 3220 additionally project from the holder 3200 and into the lumen 3206. The ribs 3220 act to create flexible ledges or compartments into which each implant 250 can be placed during loading and transportation, thus ensuring at least a minimal amount of space between each implant 250 when in the stacked orientation within the lumen 3206. The spacing can reduce scratches and other contact damage to implants 250 that can occur from implants 250 rubbing against each other when in the holder. The alignment ribs 3220 are flexible circular rings that are spaced evenly along the lumen 3206, and each rib has an opening therethrough formed along a longitudinal axis A4 of the holder 3200. As such, as each surgical implant 250 is received in or removed from the lumen 3206, each alignment rib 3220 flexes and bends as each implant 250 moves past. The rubs 3220 thus also control or regulate the rate of implant removal and prevents the implants 250 from being spilled or dumped from the holder 3200. The ribs 3220 can be made of the same material or different material than the holder 3200, and flexibility of the ribs 3220 can be varied by changing the materials used and the size, shape, and/or thickness of the ribs 3220.

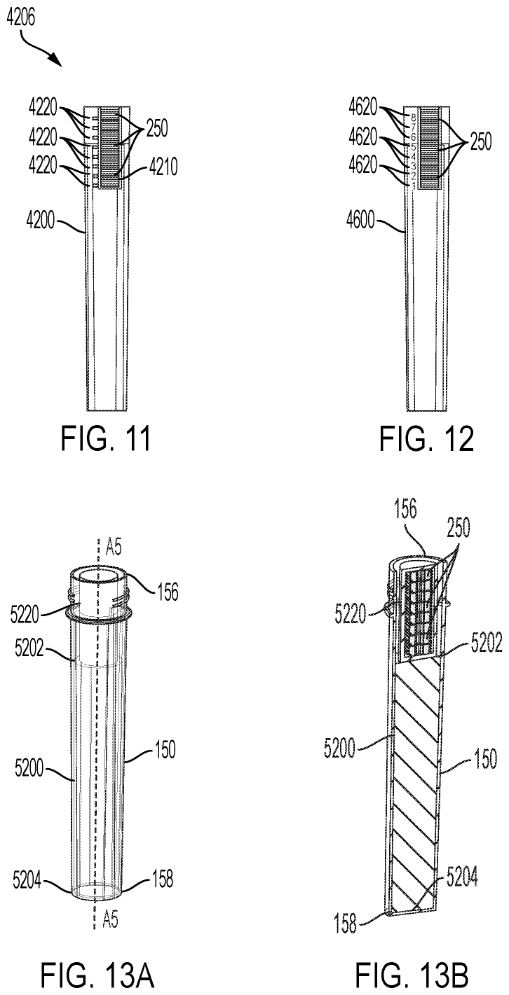

Some embodiments of the surgical holders can have markings on exterior surfaces thereof to assist the user by providing information about the surgical implants held therein, such as indicating how many implants are in a lumen; the type, size, and/or model number of the implants held therein; etc. For example, FIG. 11 illustrates one embodiment of a surgical holder 4200, similar to holder 200, that has tick marks or notches 4220 spaced along an edge of a window 4210 in the holder 4200. When a plurality of surgical implants 250 are loaded in a lumen 4206 of the holder 4200, a user can use the tick marks 4220 to count how many implants 250 are present. A similar embodiment of a surgical holder 4600 is illustrated in FIG. 12 and has numerical values 4620 instead of tick marks.

As noted above, various embodiments of the surgical holder can have a number of different shapes and sizes to maintain a position and orientation of implants within the inner tube 150. For example, FIGS. 13A and 13B illustrate another embodiment of a surgical holder 5200. The illustrated holder 5200 has first and second ends 5202, 5204 and a generally cylindrical shape that fills a majority of the lumen of the inner tube 150. While the holder 5200 maintains an orientation of the surgical implants 250 within the inner tube 150, similar to the surgical holder 200, the plurality of implants 250 are packaged in a cylindrical cartridge 5220 that can be used with a variety of different spinal surgical instruments. Because the cartridge 5220 already maintains the stacked arrangement of the implants, the holder 5220 thus acts to maintain the overall orientation of the cartridge 5220 in the inner tube 150 rather than directly contacting the implants 250. When inserted into the inner tube 150, the holder 5200 extends from the closed end 158 of the inner tube 150 and toward the open end 156. However, the holder 5200 does not extend entirely to the open end 156 of the inner tube 150, leaving a cavity between the first end 5202 of the holder 5200 and the cap 162 of the inner tube 150 when closed. The cartridge 5220 with the implants 250 can be held in the cavity formed between the first end 5202 of the holder 5200 and the cap 162 of the inner tube 150 along a longitudinal axis A5 of the holder 5200 such that the orientation and position of the implants 250 within the cartridge 5220 are maintained during transportation. While the illustrated cartridge 5220 is cylindrical and encloses the plurality of implants 250 on all sides, in other embodiments, the shape and size of the cartridge can vary depending on the surgical implants disposed therein and the surgical instruments used with the cartridge.

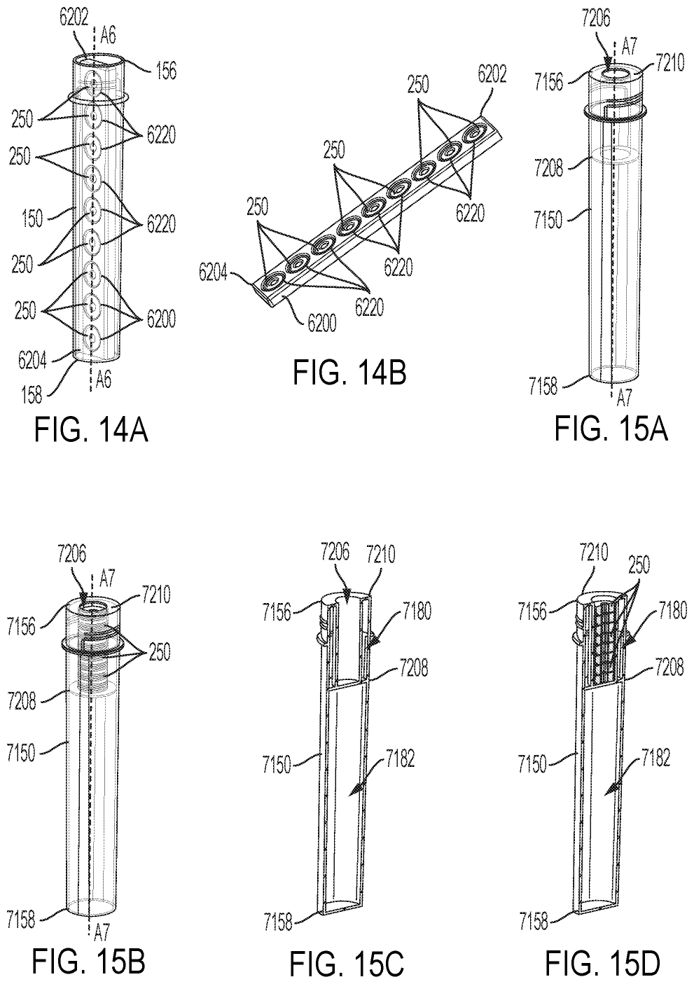

In still other embodiments of the surgical holder, the holder can function similarly to set screw caddies currently used in various spinal surgeries while still maintaining an orientation of any implants during transportation. For example, FIGS. 14A and 14B illustrate a surgical holder 6200 that has a rectangular prism structure. The holder 6200 has first and second ends 6202, 6204 and extends between the ends 156, 158 of the inner tube 150 when disposed therein, similar to surgical holder 200. The cap 162 of the inner tube 150 abuts against the first end 6202 of the holder 6200 when closed such that the orientation and position of the holder 6200 is maintained within the inner tube 150 during movement. However, a plurality of cavities 6220 are formed within a sidewall of the holder 6200 and are spaced along a longitudinal axis A6. The cavities 6220 receive the plurality of surgical implants 250 therein such that each implant 250 is received in a corresponding cavity 6220 that is sized and shaped to secure one implant 250 in a frictional engagement. As such, the implants 250 are spaced apart from one another along the axis A6 and do not come into contact with each other during transportation, helping to reduce possible damage to the implants 250. Furthermore, a user can remove the holder 6200 from the inner tube 150 during an operation and lay the holder 6200 flat on a working surface to extract the implants 250 from the holder 6200, as needed. The holder 6200 can thus function similarly to a set screw caddy used currently to load set screws while reducing the sterilization and storage requirements caused by a caddy.

In other embodiments, structures of the inner tube can be varied to either replace or entirely eliminate the need for a surgical holder, and the structure of the inner tube can be used to maintain an orientation of surgical implants therein. FIGS. 15A-15D illustrate an embodiment of an inner tube 7150 with an outer structure similar to inner tube 150 and an inner structure similar to holder 200. The inner tube 7150 can thus function both as an inner tube that is inserted into the outer tube 100 to maintain sterilization of any implants therein and as a holder to engage with and maintain an orientation of the implant(s) during transportation, simplifying use and deployment of the implants.

For example, the inner tube 7150 has an open end 7156, a closed end 7158, and threading that can engage a cap, such as the cap 162, to close the inner tube 7150. The inner tube 7150 additionally has an inner structure used to receive and engage one or more surgical implants 250. Within the inner tube 7150, a lumen 7206 is formed along a longitudinal axis A7 of the tube 7150 that extends from the open end 7156 and toward the closed end 7158. The lumen 7206 is sized and shaped to receive one or more surgical implants therein in a stacked orientation, and a length and size of the lumen 7206 can be varied to accommodate different numbers and types of implants 250, similar to the lumen 206 discusses above. The illustrated embodiment has voids or cavities formed between the lumen 7206 and outer surfaces of the tube 7150. For example, a circular platform 7208 is formed within the tube 7150 at an end of the lumen 7206 opposite the open end 7156 of the tube 7150 to create a bottom surface of the lumen 7206 onto which the implants 250 can be received, and a circular top or disc 7210 is formed at the open end 7156 with an opening therethrough for the lumen 7206 to close the surface around the lumen 7206. As such, a first void or cavity 7180 is formed between sidewalls of the lumen 7206 and outer sidewalls of the inner tube 7150 and between the open end 7156 and the platform 7208, and a second void or cavity 7182 is formed between the platform 7208 and the closed end 7158. However, in other embodiments, the voids can be filled with material such that the interior structure of the inner tube 7150 is solid, similar to holder 200.

Additionally, at least part of the inner tube 7150 can be made of transparent material to allow visualization of the implants therein. All components in the illustrated tube 7150 can be manufactured as one structure, and the tube 7150 can be manufactured using the same or similar injection molding machinery as used for the inner tube 150. For example, certain gates can be opened during injection molding to allow material fluid flow into specific mold sections for the inner tube 7150 while other gates are shut to specific mold sections for the inner tube 150. In other embodiments, however, various components can be made separately and then combined, and similar manufacturing processes and materials can be used as in the inner tube 150, discussed above.

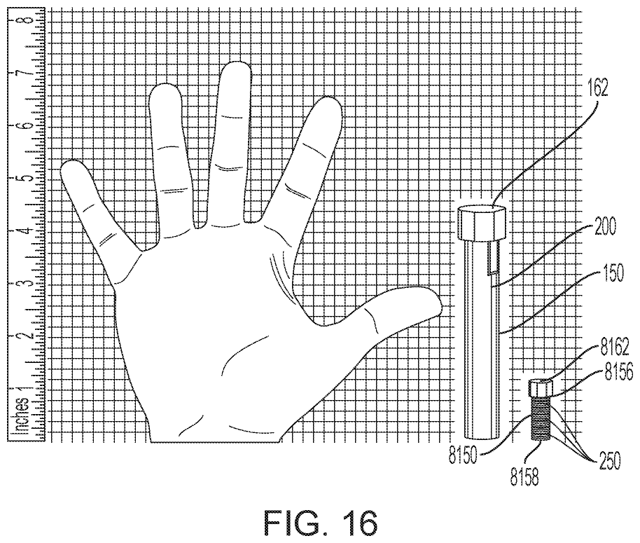

FIG. 16 illustrates another embodiment of an inner tube 8150 that is sized and shaped similar to lumen 206 to receive and maintain an orientation of the plurality of set screws 250 within a lumen therein without any intervening structures. The inner tube 8150 has an open end 8156, a closed end 8158, and threading that can engage a cap 8162 to close the tube 8150 and maintain a sterile environment therein. The illustrated inner tube 8150 is sized and shaped to receive 8 set screws in the stacked orientation, and FIG. 16 illustrates one example of the possible size difference between the inner tube 150 and holder 200 compared to the inner tube 8150 for holding the same number of implants. As such, the reduced size and complexity of the inner tube 8150 can simplify transportation and storage of the implants 250. However, in other embodiments, the size and the shape of the inner tube can be varied depending on the number and type of implants being used. Additionally, the illustrated inner tube 8150 is also made of at least some transparent material to allow visualization of the implants 250 therein, and in some embodiments, similar manufacturing processes and materials can be used as in the inner tube 150.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A surgical holding system, comprising:
an outer tube having a sealed first outer end and an open second outer end, the outer tube having a removable outer cap configured to selectively seal the second outer end;
an inner tube configured to be disposed within the outer tube, the inner tube having a sealed first inner end configured to abut against the outer cap of the outer tube, the inner tube having an open second inner end and a removable inner cap configured to selectively seal the second inner end;
a plurality of surgical implants; and
a holder configured to be disposed within the inner tube and having a longitudinal axis, the holder being configured to engage the plurality of surgical implants such that the surgical implants are held stationary within and relative to the inner tube.

2. The system of claim 1, wherein the plurality of surgical implants are positioned along the longitudinal axis of the holder.

3. The system of claim 1, wherein the holder has at least one cavity formed therein and configured to receive the plurality of surgical implants.

4. The system of claim 3, wherein the holder has a window extending through a sidewall of the holder from an outer surface of the holder to the at least one cavity such that the plurality of surgical implants is visible through the window.

5. The system of claim 4, wherein the holder has a plurality of markings on the outer surface thereof extending along an edge of the window, and the plurality of markings are configured to provide information about the plurality of surgical implants held therein.

6. The system of claim 3, wherein the at least one cavity comprises a cylindrical cavity and extends along the longitudinal axis of the holder.

7. The system of claim 6, wherein the holder has an engagement post extending along the longitudinal axis and into the cylindrical cavity, and the engagement post is configured to extend through an opening in each of the plurality of surgical implants when the plurality of surgical implants are in engagement with the holder.

8. The system of claim 7, wherein the engagement post has alignment features thereon, and the alignment features are configured to orient the plurality of surgical implants in a shared direction.

9. The system of claim 7, wherein the engagement post is configured to be at least partially retractable out of the cylindrical cavity and into the holder during loading and unloading of the plurality of surgical implants.

10. The system of claim 6, wherein the holder has a plurality of spacing lips extending into the cylindrical cavity, and each of the plurality of spacing lips is configured to prevent the plurality of surgical implants from contacting each other.

11. The system of claim 3, wherein the at least one cavity comprises a plurality of cavities, and each of the plurality of cavities is configured to receive one of the plurality of surgical implants therein.

12. The system of claim 1, wherein the outer tube, the inner tube, and the holder are configured to be coaxial with each other along the longitudinal axis of the holder when the holder is disposed within the inner tube and the inner tube is disposed within the outer tube.

13. An implant holding system, comprising:
an outer tube having a sealed first outer end and an open second outer end, the outer tube having a removable outer cap configured to selectively seal the second outer end;
an inner tube configured to be disposed within the outer tube, the inner tube having a sealed first inner end configured to contact the outer cap of the outer tube, the inner tube having an open second inner end and a removable inner cap configured to selectively seal the second inner end;
a plurality of surgical implants; and
a holder configured to be disposed within the inner tube, the holder having a longitudinal axis extending between first and second holder ends, and a lumen extending at least partially therethrough from the second holder end toward the first holder end, the lumen being configured to receive the plurality of surgical implants therein in an engagement fit such that an orientation of each of the plurality of surgical implants relative to the holder is maintained when the plurality of surgical implants are received in the lumen.

14. The system of claim 13, wherein the plurality of surgical implants comprise one of a plurality of set screws or a plurality of nuts.

15. The system of claim 13, wherein an engagement post extends along the longitudinal axis of the holder at least partially into the lumen from the first holder end toward the second holder end, and the engagement post is configured to engage each of the plurality of surgical implants.

16. The system of claim 13, wherein the plurality of surgical implants are positioned along the longitudinal axis of the holder.

17. The system of claim 13, wherein the holder has a window extending through a sidewall of the holder from an outer surface of the holder to the lumen such that the plurality of surgical implants is visible through the window.

18. A surgical method, comprising:
removing a sealed cover from an outer tube, wherein the outer tube houses an inner tube, a surgical holder is disposed within the inner tube, and a plurality of surgical implants are held in a first orientation within a lumen of the surgical holder relative to a longitudinal axis of the surgical holder through direct engagement between the surgical holder and each of the plurality of surgical implants, and wherein an interior of the outer tube, the inner tube, the implant holder, and the plurality of surgical implants are sterile;
passing the inner tube into a sterile field without contaminating an exterior surface of the inner tube;
removing a cover from the inner tube within the sterile field; and
retrieving at least one of the plurality of surgical implants from the surgical holder in the inner tube within the sterile field, the plurality of surgical implants remaining in the first orientation during retrieval through engagement with the surgical holder.

19. The method of claim 18, further comprising visually inspecting an orientation of the plurality of surgical implants through the outer tube, the inner tube, and the surgical holder during removal of the sealed cover from the outer tube.

20. The method of claim 18, wherein retrieving at least one of the plurality of surgical implants includes inserting a loading device at least partially into the inner tube and loading the at least one set screw onto the loading device.
